(19) United States
(12) Patent Application Publication
(10) Pub. No.: US 2025/0095861 A1
(43) Pub. Date: Mar. 20, 2025

(54) COLLABORATIVE, CONTINUOUS, AND COMPREHENSIVE RISK MANAGEMENT SYSTEM

(71) Applicant: Intellectual Frontiers LLC, Silver Spring, MD (US)

(72) Inventor: Shahid N. Shah, Silver Spring, MD (US)

(73) Assignee: Intellectual Frontiers LLC, Silver Spring, MD (US)

(21) Appl. No.: 18/963,451

(22) Filed: Nov. 27, 2024

(51) Int. Cl. G16H 50/30; G16H 10/60

(52) U.S. Cl. CPC G16H 50/30; G16H 10/60

(57) ABSTRACT

A system and method for generating a continuous and adaptive risk profile through multi-source data integration is provided. The system includes a processor and a memory operatively connected to the processor. The memory stores instructions that, when executed by the processor, cause the system to retrieve and integrate structured data, semi-structured data, and unstructured data. The processor is further configured to generate the risk profile by executing a real-time risk scoring algorithm. The processor is further configured to monitor and recalibrate the risk profile continuously at predefined time intervals. The system is coupled to a computer-implemented risk mitigation apparatus that is configured to continuously adapt interventions in response to the continuous and dynamic risk profile.

### BACKGROUND

### Technical Field

The embodiments herein generally relate to systems and methods for real-time, continuous risk assessment and mitigation in healthcare, particularly through multi-source data integration and adaptive patient risk profiling using machine learning and predictive analytics.

### Description of the Related Art

In recent years, the healthcare industry has increasingly relied on data-driven approaches for assessing and managing patient health risks. These methods try to integrate diverse data sources into patient well-being. However, conventional risk assessment and mitigation systems are often limited in their ability to continuously and adaptively assess and mitigate patient risks. They typically rely on static or periodic assessments, which fail to account for the dynamic nature of patient health, leading to delayed responses to emerging health risks.

Current systems face several challenges, including the difficulty of integrating structured, semi-structured, and unstructured data from multiple sources. There is a need of a comprehensive and continuous risk assessment and mitigation systems that integrate multi-source data-including structured data, semi-structured data, and unstructured data.

### SUMMARY

In view of the foregoing, an embodiment herein provides a computer-implemented system for generating a continuous and adaptive patient risk profile through multi-source data integration. The system includes a processor and a memory operatively connected to the processor. The memory stores instructions that, when executed by the processor, cause the system to retrieve and integrate patient data from structured data from Electronic Health Records (EHRs), semi-structured data from monitoring devices, and unstructured data from external sources. Each category of patient data is subjected to a source-specific normalization and validation process prior to integration. The processor is further configured to generate a patient risk profile by executing a real-time risk scoring algorithm. The patient risk profile is derived from the aggregated and pre-processed patient data. The processor is further configured to apply a dynamic weighting to each type of patient data. The weighting is determined by predefined contextual rules. The contextual rules incorporate reliability factor of the data source and relevance of the data to the patient's current health context in determining the weighting. The processor is further configured to adjust the applied weighting in real-time based on detected deviations from baseline values within the patient data. The processor is further configured to monitor and recalibrate the patient risk profile continuously at predefined time intervals. The frequency and timing of recalibration are dynamically adjusted based on a duration and magnitude of deviations in the patient data from baseline values.

An embodiment herein provides a computer-implemented method for generating a continuous and adaptive patient risk profile through multi-source data integration. The method includes retrieving and integrating patient data from structured data from Electronic Health Records (EHRs), semi-structured data from monitoring devices, and unstructured data from external sources, wherein each category of patient data is subjected to a source-specific normalization and validation process prior to integration. The method includes generating a patient risk profile by executing a real-time risk scoring algorithm, wherein the patient risk profile is derived from the aggregated and pre-processed patient data. The method includes applying a dynamic weighting to each type of patient data, wherein said weighting is determined by predefined contextual rules. The rules incorporate a reliability factor of the data source and relevance of the data to the patient's current health context. The method includes adjusting the applied weighting in real-time based on detected deviations from baseline values within the patient data. The method includes monitoring and recalibrating the patient risk profile continuously at predefined time intervals, wherein the frequency and timing of recalibration are dynamically adjusted based on the duration and magnitude of deviations in the patient data from baseline values.

An embodiment herein provides a computer-implemented patient risk mitigation apparatus for continuously adapting healthcare interventions in response to a dynamic multi-dimensional patient risk profile. The computer-implemented patient risk mitigation apparatus includes a risk profile acquisition interface configured to securely ingest and interpret the multi-dimensional patient risk profile from an external risk assessment engine, wherein the multi-dimensional patient risk profile includes an aggregated clinical and social risk score to collectively indicate a dynamic patient's real-time health risk status. The computer-implemented patient risk mitigation apparatus includes an adaptive treatment protocol synthesizer in operative connection with the risk profile acquisition interface, configured to autonomously generate and recalibrate patient-specific one or more treatment protocols in real-time in response to variations in the received multi-dimensional patient risk profile. The one or more patient treatment protocols are specifically targeted at mitigating identified one or more risk factors indicative through the multi-dimensional patient risk profile. The computer-implemented patient risk mitigation apparatus includes a machine learning-driven next-best-action computational module, in communication with the adaptive treatment protocol synthesizer, configured to analyze the multi-dimensional patient risk profile and the one or more treatment protocols, and produce one or more hyper-personalized next best actions tailored to address the identified one or more risk factors. Each of the one or more next best actions are dynamically adjusted to align with updates in the multi-dimensional patient risk profile.

### BRIEF DESCRIPTION OF THE DRAWINGS

The features of the disclosed embodiments may become apparent from the following detailed description taken in conjunction with the accompanying drawings showing illustrative embodiments herein, in which:

FIG. 1 illustrates a block diagram of a risk assessment system communicatively coupled to a plurality of data sources, in accordance with an embodiment.

FIG. 2 illustrates sub-components of the risk assessment system among other components, in accordance with an embodiment.

FIG. 3 illustrates sub-components of the risk assessment system among other components, in accordance with an embodiment.

FIG. 4 illustrates a local assistant hub 402 connected to various other components of the risk assessment system and a plurality of data sources, in accordance with an embodiment.

FIG. 5 illustrates a glucose monitoring system connected to a plurality of data sources, in accordance with an embodiment.

FIG. 6 illustrates a risk mitigation apparatus communicatively connected to the risk assessment system and the data sources, in accordance with an embodiment.

FIG. 7 illustrates various components of the risk mitigation apparatus, in accordance with an embodiment.

FIG. 8 illustrates various components and sub-components of the risk mitigation apparatus among other external tools, in accordance with an embodiment.

FIG. 9 illustrates an exemplary blockchain-configured ecosystem architecture containing one or more components of the patient risk mitigation system and the risk assessment system, in accordance with an embodiment.

FIG. 10 illustrates a representative hardware environment for practicing various embodiments.

### DETAILED DESCRIPTION

The embodiments herein and the various features and advantageous details thereof are explained more fully with reference to the non-limiting embodiments that are illustrated in the accompanying drawings and detailed in the following description.

Descriptions of well-known components are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

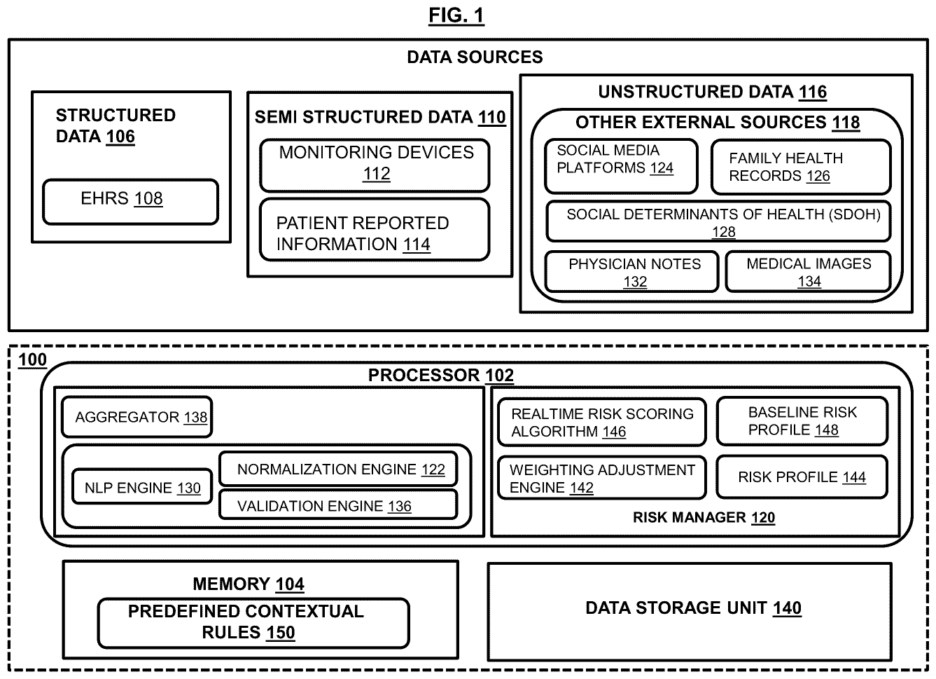

In the following detailed description, reference is made to the accompanying drawings which form a part hereof, and in which is shown by way of illustration specific embodiments in which the embodiments herein may be practiced. These embodiments, which are also referred to herein as “examples,” are described in sufficient detail to enable those skilled in the art to practice the embodiments herein, and it is to be understood that the embodiments may be combined, or that other embodiments may be utilized and that structural, logical, and electrical changes may be made without departing from the scope of the embodiments herein. Referring now to the drawings, and more particularly to FIGS. 1 through 10, where similar reference characters denote corresponding features consistently throughout, there are shown exemplary embodiments. In the drawings, the size and relative sizes of components, layers, and regions, etc. may be exaggerated for clarity.

In accordance with the disclosed embodiments, a computer-implemented system or risk assessment system or simply system 100 is provided for generating a comprehensive and adaptive risk profile through multi-source data integration. FIG. 1 illustrates an architecture and interaction of various components including the risk assessment system 100, designated with corresponding reference numerals as explained below.

The system 100 may include a processor 102 and a memory 104 operatively connected to the processor 102. The memory 104 may store instructions that, when executed by the processor 102, enable the system 100 to retrieve and integrate patient data from multiple data sources, such as structured data 106 from one or more Electronic Health Records (EHRs) 108, semi-structured data 110 from one or more monitoring devices 112 and patient reported information 114, and unstructured data 116 from one or more other external sources 118.

In the disclosed embodiments, the system 100 may be configured to retrieve and integrate the patient data from the multiple data sources, categorized into the structured data 106, the semi-structured data 110, and the unstructured data 116. Each data type undergoes a source-specific normalization and validation process before being integrated into the comprehensive and adaptive risk profile 144.

The structured data 106 may refer to highly organized data, typically originating from the one or more Electronic Health Records (EHRs) 108. The EHRs 108 may contain the patient data in a predefined format, including vital signs, medical history, laboratory test results, and physician notes. This type of data may be directly compatible with a real-time risk scoring algorithm of a risk manager 120 of the system 100 discussed hereafter, allowing for seamless integration without extensive pre-processing.

The semi-structured data 110 may originate from the one or more monitoring devices 112, such as wearable health trackers (also referred to as wearables or wearable devices), which may capture real-time patient metrics such as heart rate, blood pressure, and glucose levels. This type of data, while somewhat organized, may lack rigid structure of the structured data 106 such as the EHRs 108 and may require a pre-processing step with use of hardware and software components. The system 100 may include a normalization engine 122 to standardize this data format, to ensure that it is processed alongside the structured data. The system 100 may be equipped to handle the patient-reported information 114, which may be semi-structured and may be entered through a user interface (not shown in FIG. 1) by a user, using input methods such as voice, touch, or text, without limitations. The user interface may allow the user to manually input their data into the system 100, thereby enabling a personalized risk profile such as the comprehensive and adaptive risk profile (also referred to as risk profile interchangeably throughout this document) 144.

The unstructured data 116 may present the greatest challenge for integration, as it does not follow a predefined structure at all. Sources of the unstructured data 116 may include such as the one or more other external sources 118 (referred to as external sources or other external sources interchangeably for simplicity of description hereafter) such as social media platforms 124, and hereditary health records or family health records 126. This type of data may include Social Determinants of Health (SDoH) 128, which may be important for assessing a patient's overall risk including their social risk. The system 100 may incorporate a machine-learning-based Natural Language Processing (NLP) engine 130 that may be configured to retrieve, validate, and normalize the SDoH 128 and other form of the semi-structured data 110 and the unstructured data 116 by converting it into structured formats compatible with the real-time risk scoring algorithm 146.

For example, the system 100 may extract and analyze social media data from the social media platforms 124 for insights into a patient's behavioral and lifestyle patterns, while also retrieving the hereditary health data from the family health records 126 to assess genetic risk factors. The NLP engine 130 may be configured to transform the unstructured data 106 into a format that can be used by the system 100 to create the comprehensive and adaptive risk profile 144.

The ability of the system 100 to consolidate the structured data 106 from the EHRs 108, the semi-structured data 110 from the monitoring devices 112 such as the wearables, and the unstructured data 116 from the other external sources 118 provides a computer executable and processed unified view for users such as healthcare providers. This consolidated view may improve decision-making by presenting all relevant data types in a single interface, enabling accurate patient risk assessments. A dynamic weighting and real-time recalibration may be provided to further ensure that all data types are appropriately accounted for in the risk profile 144 to continuously update the risk profile 144 as new data is received by the system 100.

The integration of these diverse data sources may allow the system 100 to generate the comprehensive and adaptive risk profile (interchangeably referred to as risk profile or simply profile without limitations) 144 that may reflect a current health status of a patient, taking into account not only clinical data but also social, behavioral, and hereditary factors. This may provide a holistic approach to patient care and risk management.

Each category of the patient data may be processed by the source-specific normalization engine 122 also referred to simply as the normalization engine 122 and a validation engine 136 prior to integration. The normalization engine 122 may be configured to adjust data formats to a uniform standard, while the validation engine 108 may be configured to verify integrity and reliability of the incoming data.

Once the data is validated and normalized, an aggregator 138 may aggregate the structured data 106, the semi-structured data 108, and the unstructured data 116 into a unified format for further processing. The integrated data may be stored in a data storage unit 140 for use in generating the patient risk profile 144.

The system 100 may execute the real-time risk scoring algorithm 146 from the risk manager 120 to generate the risk profile 144. The real-time risk scoring algorithm 146 may derive the risk profile 144 by analyzing the aggregated patient data stored in the data storage unit 140. The risk profile 144 may be continuously updated as new data becomes available.

The system 100 may apply a dynamic weighting to each type of patient data, determined by predefined contextual rules (also referred to as contextual rules interchangeably without limitations) 150. The contextual rules may incorporate a reliability factor of a specific data source and a relevance of respective data to the patient's current health context. The system 100 may include a weighting adjustment engine 142 configured to recalibrate the weighting in real-time based on deviations from baseline values within the patient data. For instance, if certain data points exhibit deviations, the system 100 may increase or decrease their contribution to an overall risk score (and risk profile) based on a magnitude and duration of the deviations.

The risk manager 120 of the system 100 may continuously track the risk profile 144 and detect changes in the patient data over time. The risk manager 120 may recalibrate the risk profile 144 at predefined time intervals. However, the risk manager 120 may dynamically adjust recalibration frequency based on the duration and the magnitude of the deviations detected by the risk manager 120. If the deviations persist beyond a threshold, the risk manager 120 may increase the recalibration frequency to maintain an accurate and timely risk profile 144.

In an embodiment, the system 100 may retrieve the patient data from the EHRs 108, the monitoring devices 112, and the other external unstructured data sources 118, such as physician notes 132 or medical images 136. Each data stream may undergo the aforementioned normalization and validation processes to ensure that only reliable and consistent data is used for patient risk assessment.

The real-time risk scoring algorithm 146 contained within or communicatively coupled with the risk manager 120 may be configured to generate a baseline risk profile 148 and update it dynamically as new data flows into the system 100. The risk profile 144 may be executed by the real-time risk scoring algorithm 146, such that the patient risk profile 144 is derived from the aggregated and pre-processed patient data. The weighting adjustment engine 142 may evaluate the relevance of each data stream in real-time and assign the weighting or dynamic weighting factor that may reflect the reliability of the source and the current health context of the patient.

If the patient data deviates from the established baseline values, the risk manager 120 may increase the frequency of risk profile updates. For example, if the semi-structured data 110 from the monitoring devices 112 indicates an irregular heart rate, the system 100 may adjust the weighting or weight of this data accordingly and recalibrate the patient's risk profile 144 more frequently to reflect this change.

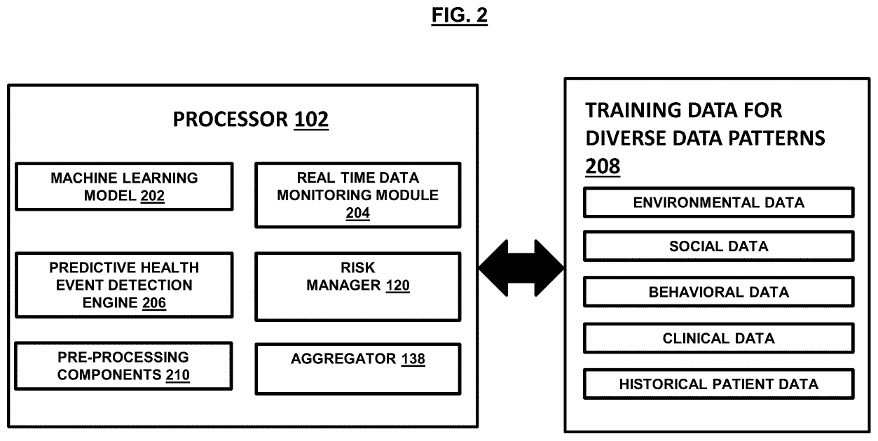

FIG. 2 illustrates the processor 102 or hardware-based AI processing unit 102 or simply processing unit 102 (used interchangeably) for performing the source-specific normalization and validation for the unstructured data 116 and the semi-structured data 110 using a machine learning model 202 among other tasks that the processor 102 may perform based on training data for diverse data patterns. The machine learning model 202 may be trained on diverse data patterns, including such as environmental, social, and behavioral data, clinical data, without limitations, and may adapt the validation and the normalization process based on the contextual relevance of the data to the patient's current health status. The machine learning model 202 may be executed by the hardware-based AI processing unit 102, allowing for real-time data processing and validation.

The unstructured data 116 may include such as the Social Determinants of Health (SDoH) 128, which may be retrieved from the other external sources 118 such as the social media platforms 124. A machine-learning-based natural language processing (NLP) engine such as the NLP engine 130 may process this data by converting the unstructured data 116 into structured formats that are compatible with the system's real-time risk scoring algorithm 146. The NLP engine 130 may assign the reliability factor to the unstructured data 116 based on its accuracy and timeliness. The reliability factor may be adjusted dynamically as the system 100 evaluates data integrity and relevance in real-time.

The machine-learning-based NLP engine (also referred to as simply the NLP engine interchangeably through this document) 130 may include one or more modules for parsing and extracting key entities from the unstructured data 116 using named entity recognition (NER) and other NLP techniques without limitations. The machine learning model 202, that may be trained on labeled datasets, may evaluate the accuracy of the extracted data by comparing it against known, validated sources. Timeliness may be determined by analyzing timestamps and comparing them to current patient health events.

The reliability factor may be calculated using a weighted combination of the accuracy and timeliness. The system 100 may use this reliability factor to adjust the weighting of the unstructured data 116 and the semi structured data 110 along with the structured data 106 when calculating the risk profile 144.

The processor 102 may include a real-time data monitoring module 204, as illustrated in FIG. 2, which may continuously track and collect the patient data from the monitoring devices 112, the other external sources 118, and the EHRs 108. The real-time data monitoring module 204 may be integrated with the processor 102 or may be communicatively coupled separately such that the processor 102 may dynamically adjust the risk profile 144 in response to the detected deviations from the baseline values as discussed in FIG. 1. The processor 102 may be configured to recalibrate the risk profile 144 without human intervention, ensuring comprehensive and continuous monitoring with real-time updates.

In an embodiment, the processor 102 may include a predictive health event detection engine 206. The predictive health event detection engine 206 may be implemented on the processor 102 or may be communicatively coupled to the processor 102 and continuously analyze patient data trends to detect early warning signs of adverse health events. The predictive health event detection engine 206 may generate alerts when it predicts an upcoming health event. The predictive health event detection engine 206 may use the machine learning model 202 trained on historical patient data or training data 208 to recognize one or more critical health deviations such as the deviations discussed above. The processor 102 may contain more components such as shown in FIG. 1, in some embodiments.

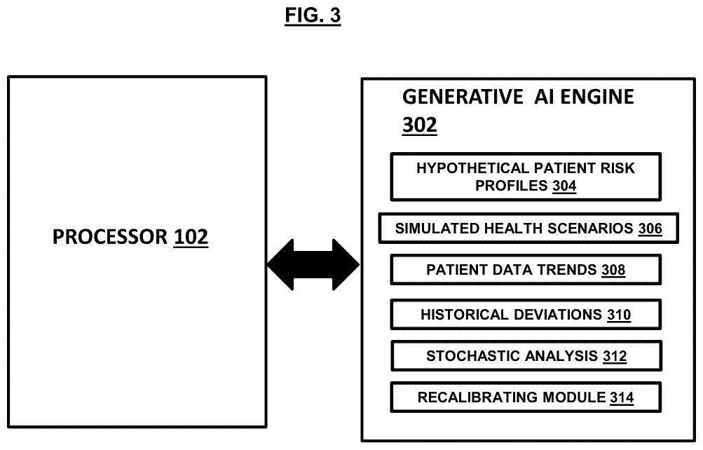

FIG. 3 illustrates a generative AI engine 302 for simulating health scenarios. In accordance with some embodiments, the system 100 may include the generative AI engine 302, as illustrated in FIG. 3 that may be operatively connected to the processor 102. The generative AI engine may generate one or more hypothetical patient risk profiles 304 by simulating one or more health scenarios called simulated health scenarios 306 or simply simulations referred interchangeably throughout this document. These simulations 306 may be based on one or more of patient data trends 308, historical deviations 310, and stochastic analysis 312 of evolving health patterns across the structured data, the semi-structured data, and the unstructured data collected by the system 100. The generative AI engine 302 may be configured to enable healthcare providers to prepare for potential health risks by modeling various health trajectories.

The generative AI engine 302 may be configured to continuously adjust one or more parameters of the simulations based on real-time data inflows. As the patient data, such as vital signs from the monitoring devices 112 or unstructured behavioral inputs from such as the social media platforms 124 etc, is integrated into the system 100, the generative AI engine 302 may recalibrate its models to reflect evolving health trajectories with the use of a recalibrating module 314. This adaptive recalibration may be enabled by the system's real-time risk scoring algorithm 146, which may assign the dynamic weightings to different data types based on their relevance and reliability. The generative AI engine 302 may provide healthcare providers with predictive insights that may evolve alongside the patient's health status, thereby improving the ability of the system 100 to predict potential adverse health events before they occur.

The generative AI engine 302 may not only simulate linear health trends but may also simulate non-linear and stochastic health events, which may be common in patients with complex, multifactorial conditions. The generative AI engine 302 may incorporate stochastic modeling techniques to predict unexpected deviations in patient health trajectories, such as sudden exacerbations of chronic conditions or the onset of acute health crises. The generative AI engine 302 may use the historical deviations as reference points and cross-analyze them with the real-time patient data to identify patterns that may indicate impending health risks. This may ensure that the risk profiles 144 generated by the system 100 are not merely reflections of past and present data but are projections of future health developments that may account for the unpredictable events.

The integration of the structured data 106, the semi-structured data 110, and the unstructured data 116 in the generative AI engine 302 may provide comprehensiveness and granularity to risk simulations in real-time. The structured data 106, such as the EHRs 108, may provide a view of the patient's medical history, while the semi-structured data 110 from the monitoring devices 112 such as the wearable devices may offer real-time physiological insights. The inclusion of the unstructured data 116, such as the social determinants of health (SDoH) 118 and the behavioral insights from the other external sources 118, may allow the generative AI engine 302 to simulate the health scenarios that may take into account many other external factors that could influence patient outcomes but may not be covered in the EHR data and the structured and the unstructured sources 106, 110, and 116. This holistic approach to the data integration may allow that the simulated risk profiles or the hypothetical patient risk profiles 304 generated by the system 100 are not only clinically relevant but also contextually accurate, providing a more complete picture of the patient's potential health future.

The generative AI engine 302 of the system 100 may be particularly advantageous in its application for personalized healthcare, where tailored simulations may be essential for patient-specific health planning. The ability of the generative AI engine 302 to simulate the future health scenarios based on individual patient data trends and contextual factors may allow healthcare providers to anticipate future health risks and tailor preventive interventions accordingly. The system 100 may improve the ability of healthcare professionals to make informed, data-driven decisions that could significantly improve patient outcomes.

In various embodiments, the patient data retrieved by the system 100 may comprise one or more computer-executable files. These files may contain the structured data 106, the semi-structured data 110, and the unstructured data 116 of various types, including patient health data from the EHRs 108, the SDoH data 128 from the other external sources, the data from the monitoring devices 112 such as the wearable devices that monitor health metrics, the patient-reported information 114 entered through the user interface, the social media data platforms 124 analyzed for lifestyle insights, and the hereditary health data or family health records 126. Each file may be retrieved by a secure data retrieval device such as the aggregator 138 as shown in FIGS. 1 and 2 and processed by the processor 102 to normalize, validate, and integrate the patient data for real-time risk profile generation.

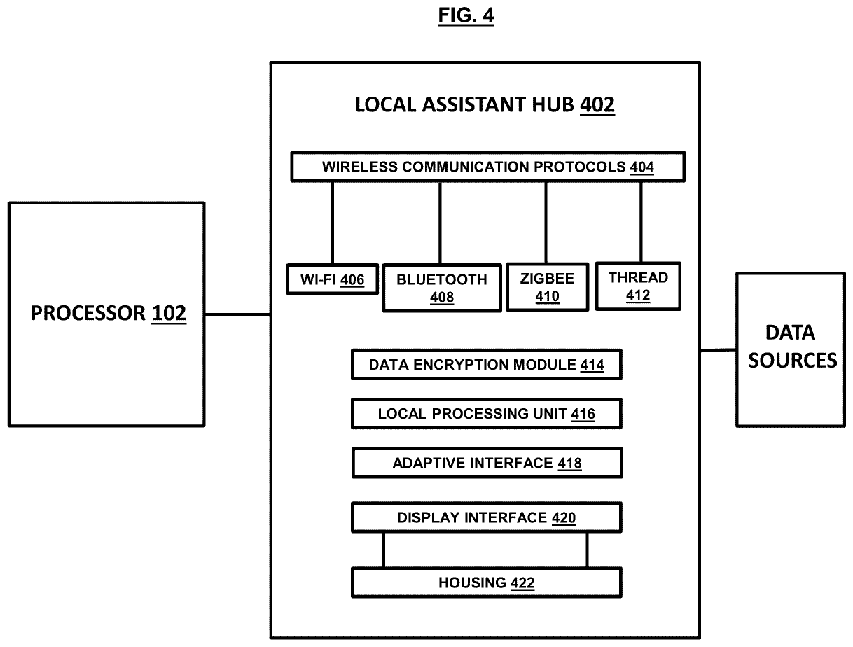

FIG. 4 illustrates a local assistant hub 402 that may be communicatively coupled to the processor 102. In an embodiment, the local assistant hub 402 may be configured with a modular hardware architecture. The local assistant hub 402 may be configured to support multiple wireless communication protocols 404, including such as Wi-Fi 406, Bluetooth 408, ZigBee 410, and Thread 412, enabling seamless integration of the EHRs 108, the monitoring devices 112, and the other external sources 118 without requiring hardware upgrades. The local assistant hub 402 may further include a data encryption module 414 for securing local storage and transmission of the patient data. The data encryption module 414 may be configured to operate without continuous connection to an external server, ensuring patient data privacy.

The local assistant hub 402 may further include a local processing unit 416, as illustrated in FIG. 4, for performing on-device data analytics and real-time risk calculations. This may allow processing of the data locally, which allows the system 100 to reduce latency and minimize reliance on cloud-based processing for continuous risk monitoring. This feature is particularly important in scenarios where low-latency responses are critical to patient health management.

In an embodiment, the system 100 may support data integration from the various monitoring devices 112, including heart rate monitors, continuous glucose monitors, and sleep tracking devices. Each device may communicate with the local assistant hub 402 using a device-specific protocol and transmit the patient data in real-time. This may ensure that the system 100 maintains up-to-date patient health metrics for generating an accurate risk profile.

In an embodiment, the local assistant hub 402 may include an adaptive interface 418, as shown in FIG. 4, for receiving the patient-generated information via voice, touch, or text input. This may allow patients to manually enter additional data into the system 100 for integration into their risk profile 144. The adaptive interface 418 may be equipped with natural language processing (NLP) capabilities, which may interpret the patient-generated information and translate it into actionable data points.

The local assistant hub 402 may further include a display interface 420 for providing real-time feedback on the patient risk profile 144. The display interface 420 may display aggregated patient data, including the structured data 106 from the EHRs 108, the semi-structured data 110 from the monitoring devices 112, and the unstructured data 116 from the other external sources 118, in a consolidated view for interpretation by a user. The local assistant hub 402 may include a housing 422 containing the display interface 420 for providing the real-time feedback on the patient risk profile 144.

As illustrated in various figures, the system 100 may be designed to scale securely by integrating new data sources and modules as required. The modular hardware design may allow for seamless upgrades without disrupting the existing system.

In an embodiment, the system 100 may incorporate genomic data into the risk profile generation. The genomic data may be retrieved from a database of genetic markers and added to the existing sources of the patient data. The genomic data may be processed through the normalization and validation process as the other structured data. The real-time risk scoring algorithm 146 may be adapted to include genomic risk factors that may predispose the patient to certain medical conditions, thus providing a more personalized and precise risk profile. The weighting may be applied to the genomic data to dynamically adjust based on the contextual rules, which consider the relevance of the genetic information in the context of the patient's current health condition. This may be particularly useful in precision medicine applications, where individual genetic differences play a key role in determining health risks and treatment plans. Incorporating genomic data can allow healthcare providers to generate a more comprehensive and personalized risk profile and update it in real time, leading to better-targeted interventions and preventive measures.

In an embodiment, the system 100 may be adapted for use in remote patient monitoring (RPM) programs, where the patient data may be continuously collected from the wearable devices and other health monitoring equipment. The local assistant hub 402 may be configured with an expanded range of wireless protocols to support integration with the various RPM devices. The real-time data monitoring module 204 as discussed above may continuously track the patient's vital signs and updates the risk profile 144 based on the data received from the monitoring devices 112.

In remote care scenarios, the ability of the system 100 to autonomously recalibrate the patient's risk profile 144 in real-time, without requiring human intervention, may provide a comprehensive and continuous risk profiling of patients. Alerts may be generated by the predictive health event detection engine 206 that may be sent to healthcare providers or family members for timely intervention. This may reduce the need for frequent hospital visits and allows for continuous monitoring of high-risk patients in the comfort of their own homes.

The system 100 may be scaled for population health management, where aggregated data from multiple patients may be used to identify trends and predict health risks across a large population. In embodiments, the system 100 may retrieve the structured and the unstructured data from the diverse sources such as the EHRs 108, public health databases, social media platforms 118, and wearable devices. The system 100 may apply the same source-specific normalization and validation process, but the risk profiles 144 generated may be analyzed at both the individual and population levels. This may help for public health agencies and healthcare organizations that need to monitor population health in real time. Healthcare providers and social care networks can identify emerging health trends and allocate resources more efficiently to address at-risk groups by generating aggregated risk profiles such as the risk profile 144. This may also support health interventions on a broader scale, enabling healthcare organizations to implement preventative measures before a large-scale health crisis occurs.

In an embodiment, the system 100 may serve as an AI-driven decision support tool for healthcare providers. The generative AI engine 302 may simulate the health scenarios 306 based on the real-time patient data trends 306 and the historical health data such as the historical deviations 308 without limitations. These simulations may help healthcare providers predict potential health outcomes and decide on the best course of action to mitigate risks. The system 100 may present the healthcare provider with multiple risk scenarios based on the patient's current risk profile and the simulated future health trajectories. This may allow the healthcare providers to assess the potential risks of different treatment plans and choose the most effective one. This may help in complex medical cases where the patient's health condition may rapidly change, requiring dynamic and informed decision-making.

In an embodiment, the system 100 may be fully integrated with existing hospital information systems (HIS), allowing for seamless data exchange between two platforms. The system 100 may retrieve patient data from the HIS, which may store a variety of the structured data 108 and the semi-structured data 110 and the unstructured data 116. The system 100 may process the data using the normalization, validation, and risk scoring algorithms, but the integration with the HIS allows for a more holistic view of the patient's health.

This may support real-time monitoring and risk assessment within hospital settings, where healthcare providers may use the system 100 to monitor multiple patients simultaneously. The system 100 may be configured to integrate with hospital alarm systems to trigger alerts for high-risk patients, ensuring timely intervention.

In an embodiment, the local assistant hub 402 may be configured with the adaptive user interface that may allow the patients to engage with the system 100 using voice, touch, or text input. The adaptive interface may use the natural language processing (NLP) to interpret the patient-generated information and integrate it into the risk profile 144. This may allow patient engagement by allowing individuals to input their symptoms, daily routines, or concerns directly into the system 100, enabling healthcare providers to incorporate this subjective information into the real-time risk scoring algorithm 146.

Patients can also view their risk profile and other relevant health data on the display interface in real-time, providing them with immediate feedback on their health status. This may help patient engagement and helps individuals take an active role in managing their health, particularly in chronic disease management scenarios.

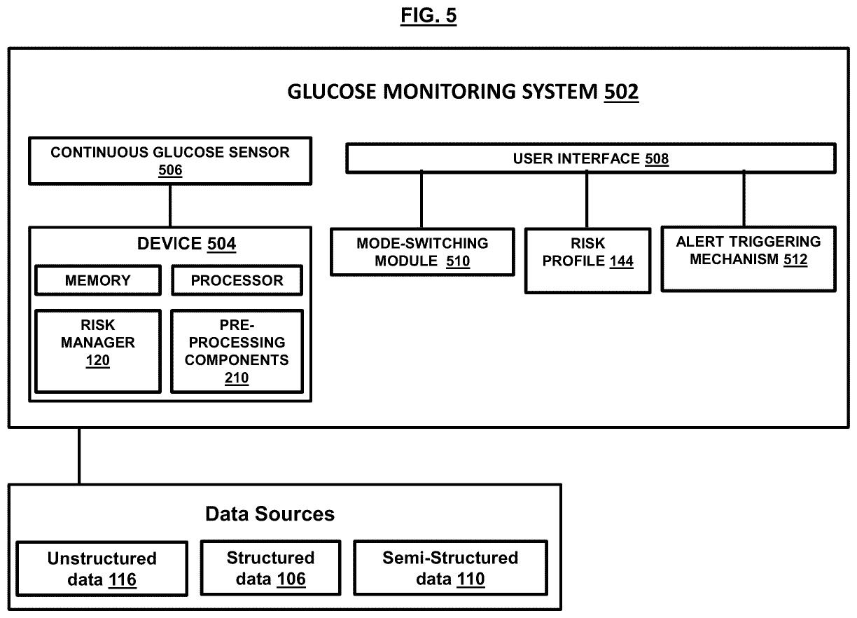

In an embodiment, the system 100 may be adapted for monitoring glucose concentration and generating a continuous and adaptive patient risk profile 144. The system 100 may be used for patients with conditions such as elevated blood glucose levels, where continuous glucose monitoring is needed for managing health risks. In embodiments, the system may be configured as a glucose monitoring system 502 such as shown in FIG. 5. The glucose monitoring system 502 may include a device 504. The device 504 may include or be coupled with a continuous glucose sensor 506. The device 504 may retrieve, process, and normalize the patient data originating from the multiple data sources, including the continuous glucose sensor or simply glucose sensor 506 and other sources as discussed already, to generate the real-time patient risk profile 144.

The continuous glucose sensor 506 may be operatively connected to or be integrated with the device 504. The glucose sensor 506 may continuously measure the glucose concentration in the patient's bloodstream and outputs a real-time data stream. The device may include the processor 102 and the memory 104 as shown in FIG. 1. The processor 102 and the memory 104 may include various components as shown in FIG. 1 such as the risk manager 120 and the pre-processing components 210 without limitations. The memory 104 may store the instructions for executing various system functions. Among other tasks as discussed in conjunction with FIGS. 1-5, the device 504 of the system 100 may perform tasks including retrieving and integrating the data stream from the continuous glucose sensor 506 with the other patient data from the various data sources, processing the data through the normalization and the validation technique, and generating the comprehensive and continuous risk profile 144 based on an output from the real-time risk scoring algorithm 140.

The integration of glucose concentration data with the other patient data may ensure that the risk profile 144 is comprehensive and adaptive. The glucose concentration data may be combined with the additional patient data such as heart rate, sleep patterns, physical activity levels, and even social determinants of health retrieved from the various data sources as discussed already.

In an embodiment, the processor 102 may dynamically adjust the weighting of the different data sources when generating the risk profile 144, as discussed in conjunction with FIGS. 1-5. The system 100 may apply a dynamic weighting algorithm by the weighting adjustment engine 142 that may assign the weight to each of the data sources, such as the glucose concentration data, activity levels, or environmental factors, based on factors like data reliability and relevance to the patient's health context. The relevance and reliability of the data sources can vary over time, and the weighting adjustment engine 142 may be configured to adapt based on real-time evaluations.

For example, during periods of rapid glucose fluctuation, the system 100 may increase the weight of the glucose concentration data in determining the risk profile 144, while reducing the weight of less critical data sources, such as physical activity levels. Conversely, if the glucose levels are stable, the system 502 may prioritize other data sources to generate a more holistic risk profile. This dynamic weighting may facilitate the most relevant and reliable data to be always prioritized in risk calculations.

The system 502 may be configured to continuously monitor the patient's glucose concentration data and other patient data for the deviations from the baseline values. If any deviations in the glucose concentration or other patient metrics exceed predefined thresholds, the system 502 may recalibrate the patient's risk profile in real-time. The recalibration process is based on the magnitude and persistence of the deviations. For example, if the patient's glucose concentration increases sharply and remains elevated for an extended period, the system 502 may dynamically adjust the risk profile 144 to reflect the heightened risk of hyperglycemia. Conversely, if the glucose levels return to the baseline within a short timeframe, the system 502 may reduce the risk level accordingly and update the risk profile 144.

The system 502 may include a user interface 508 similar to the display interface 420 that may display both the patient's real-time glucose concentration data and the continuously updated risk profile 144. As shown in FIG. 5, the user interface 508 may be designed to provide the patient or healthcare provider with a clear and concise view of the most relevant data. The glucose concentration values or data may be presented in real-time, alongside other visual indicators of whether the values fall within healthy ranges.

The user interface may also display the continuously updated risk profile 144, offering an overview of the patient's current health risks. The user interface may include additional features, such as graphical trends showing changes in the glucose concentration as well as the risk profile 144 over time, and alerts that notify the patient or healthcare provider of significant deviations in the glucose concentration or other critical health metrics or the risk profile 144.

The recalibration process may also consider other patient data, such as changes in physical activity or heart rate, ensuring that the risk profile 144 remains accurate and reflective of the patient's overall health. This real-time adjustment may allow for timely intervention and reduces the likelihood of adverse health events.

In an embodiment, the device 504 may be configured to automatically toggle between displaying the real-time glucose concentration data and the patient's risk profile 144, based on predefined conditions. The device 404 may include a mode-switching module 510 that may toggle the device 504 between a first mode for displaying the glucose concentration values or data and a second mode for displaying the risk profile 144.

The mode-switching is triggered automatically based on predefined conditions, such as significant changes in the glucose concentration data beyond the threshold or the deviations in the risk profile 144. For example, if the glucose levels spike rapidly, the device 504 may automatically switch to a glucose display mode to provide real-time feedback on the concentration levels. If the system 502 detects sustained deviations in the risk profile 144, it may toggle to a risk profile display to highlight the broader health risks that the patient faces.

This toggling function may be controlled manually through the user interface 508, allowing patients or healthcare providers to select the preferred display mode based on their current needs.

In an embodiment, the system 502 may include an alert-triggering mechanism 512 that may prompt the device 504 to automatically switch to the most critical display mode based on the changes in the glucose concentration or the deviations in the risk profile 144. The system 502 may continuously monitor the glucose data and the risk profile 144 for the deviations, and if the thresholds are breached, the system 502 may generate the alert.

The alert may take the form of a visual or auditory notification, and the system may respond by switching the display to the mode that shows the most urgent data. For example, if the glucose concentration spikes above the threshold, the device 504 may automatically switch to the glucose concentration display to provide real-time updates. If the risk profile 144 shows a significant increase in the health risks, the system 502 may toggle to the risk profile display to inform the patient or the healthcare provider of the broader health context.

In different embodiments, the system 502 may be configured for use in specific healthcare scenarios. For example, the glucose monitoring system 502 may be adapted for use by patients with chronic conditions, where continuous glucose monitoring (CGM) system 502 is essential for maintaining optimal glucose levels. The system 502 may be integrated into broader telemedicine platforms, allowing the healthcare providers to remotely monitor patients' glucose levels and overall health risks, offering timely interventions when necessary.

In embodiments, the system 502 may be used in clinical settings, where multiple patients are monitored simultaneously. The ability of the system 502 to continuously recalibrate the risk profile 144 based on the real-time patient data may be particularly beneficial in intensive care units (ICUs), where rapid changes in patient conditions must be detected early to prevent critical health events.

In embodiments, the system 502 as shown in FIG, 5 may be designed for monitoring of the glucose concentration and generating the continuous and adaptive patient risk profile 144, primarily informed by the glucose data. The system 502 may include the continuous glucose sensor 502 that may be configured to measure the glucose levels continuously and output the data stream that may correspond to these glucose concentration measurements. The system 502 may incorporate the device 504 that may be operatively connected to the continuous glucose sensor 502. The device 504 may also feature a single-point glucose monitor, which may be capable of providing discrete glucose measurements for enhanced data granularity.

The device 504 may comprise the processor 102 and the memory 104 that is operatively connected to the processor 102. The memory 104 may store instructions which, when executed by the processor 102, enable the system 502 to retrieve and integrate the continuous glucose data stream with other relevant patient data. This patient data may include the structured data 106 from the Electronic Health Records (EHRs) 108, the semi-structured data 110 from the various monitoring devices 118, and the unstructured data 116 sourced from the other external data sources 188, such as the social determinants of health or behavioral data 128.

Before integration, each category of the patient data may undergo the source-specific normalization and validation process, ensuring accuracy and relevance to the patient's health context. The system 502 may then process the aggregated data using the real-time risk scoring algorithm 146 to generate the continuous and adaptive patient risk profile 144. This profile 144 may account not only for the glucose data but also broader patient health metrics from the multiple sources.

The system 502 may dynamically adjust the weighting of the data sources in real-time. These adjustments may be based on the deviations detected from the baseline values in the glucose concentration data and other patient data, ensuring that the most relevant health information is prioritized for risk profiling.

To facilitate user interaction, the system 502 may be equipped with the user interface 508 that may display both continuous glucose concentration values from the glucose sensor 506 and discrete measurements from a single-point glucose monitor. Additionally, the user interface 508 may provide real-time updates on the patient's risk profile 144, offering a comprehensive view of the patient's current health status and any potential risks identified by the system 502.

The glucose monitoring system 502 as discussed herein is used as an exemplary embodiment. However, it must be appreciated that other wearable devices without limitations may be used to perform their respective functions of sensing their respective data from the body in direct interaction with the body such as through subcutaneous or bodily insertion or implant or through indirect communication with body through remote communication. The data fetched from the body by such wearable devices may then be used by the system 100 for risk assessment purpose.

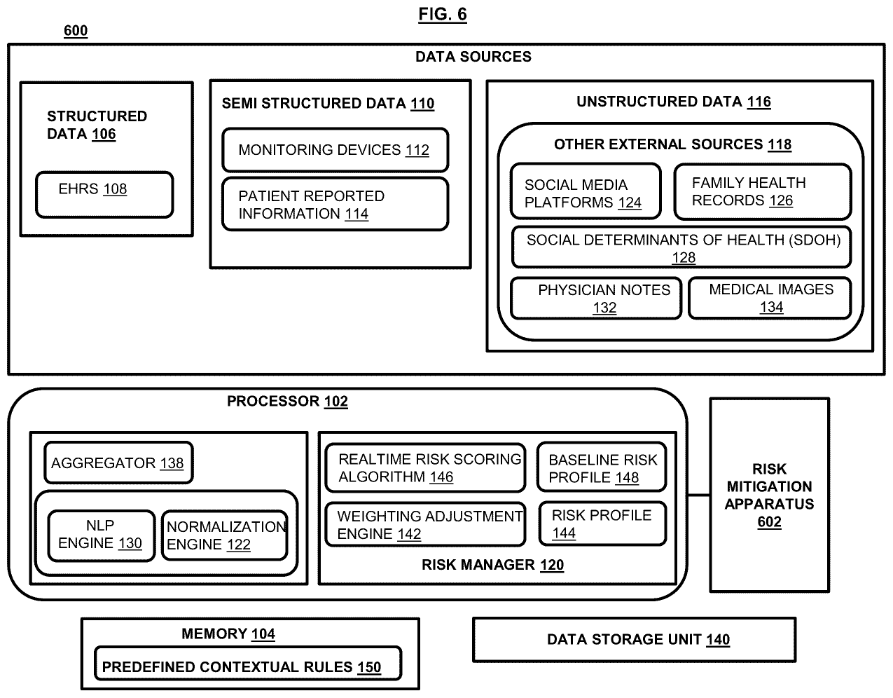

FIG. 6 illustrates a comprehensive computer-implemented system 600 for generating and continuously updating a multi-dimensional patient risk profile such as the risk profile 144 discussed above through multi-source data integration, along with an associated patient risk mitigation system 602 or patient risk mitigation apparatus 602 or simply risk mitigation system 602 (referred interchangeably without limitations) designed to dynamically adapt one or more computer executable healthcare interventions based on evolving patient-specific risk factors. The risk assessment system 100 and the risk mitigation system 602 may be comprised of various interconnected components that work collaboratively to provide a robust risk monitoring and intervention framework.

In the disclosed embodiments, the risk mitigation system 6002 may be configured to retrieve and integrate the patient data from the multiple data sources 106, 110, and 116, categorized into the structured data, the semi-structured data, and the unstructured data. Each data type undergoes the source-specific normalization and validation process before being integrated into the comprehensive and adaptive risk profile 144 as discussed above.

The Risk Mitigation System 602, integrated or communicatively coupled with the Risk Assessment System 100, may include several components that may utilize the multi-dimensional patient risk profile 144 (also referred to as risk profile interchangeably throughout this document) for the adaptive healthcare interventions.

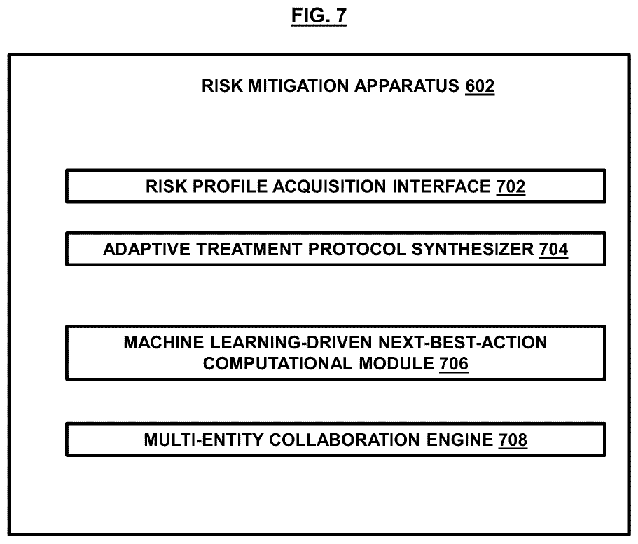

FIG. 7 illustrates various components of the risk mitigation system 602. A shown in FIG. 7, the Risk Mitigation System 602 may include a risk profile acquisition interface 702 that may be configured to securely ingest and interpret the patient risk profile 144 generated by the Risk Manager 120. The Risk Mitigation System 602 may further include an adaptive treatment protocol synthesizer 704 in operative connection with the risk profile acquisition interface 702. The adaptive treatment protocol synthesizer 704 may be configured to autonomously generate and recalibrate patient-specific one or more treatment protocols in real-time in response to variations in the received multi-dimensional patient risk profile 144. The Risk Mitigation System 602 may further include a machine learning-driven next-best-action computational module 706, in communication with the adaptive treatment protocol synthesizer 704. The machine learning-driven next-best-action computational module 706 may be configured to analyze the multi-dimensional patient risk profile 144 and the one or more treatment protocols. The Risk Mitigation System 602 may further include a multi-entity collaboration engine 708 that may be configured to facilitate secure, real-time bidirectional data exchange over a communication network among two or more entities involved in a patient's care, for synchronization and collaborative refinement of the one or more treatment protocols and the one or more next best actions. The risk assessment system 100 has been described above in conjunction with various figures.

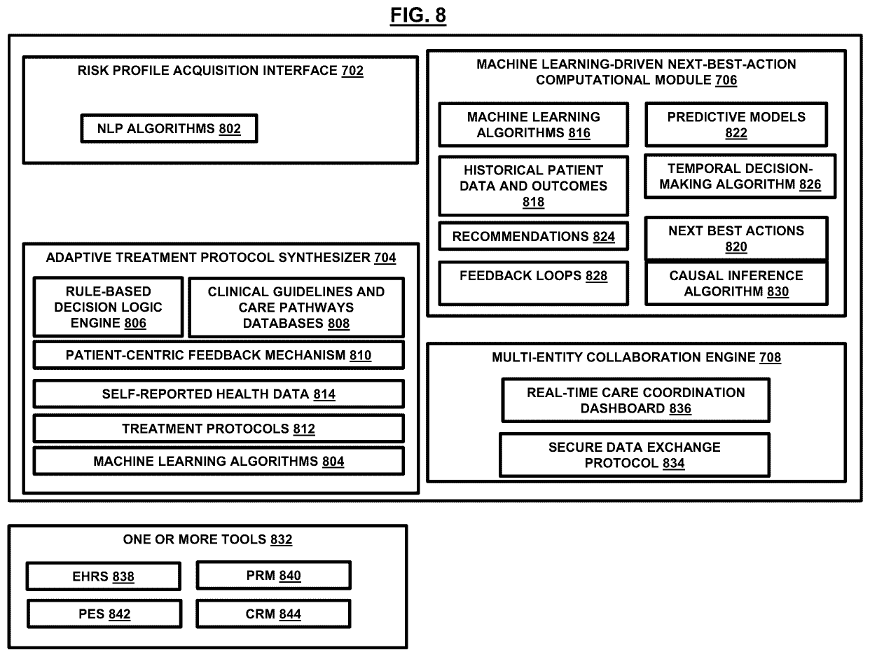

FIG. 8 illustrates a block diagram that provides detailed information about the various components of the risk mitigation system 602 and their relationships. Referring to FIGS. 6, 7, and 8, the risk mitigation system 602 is discussed herein.

In various embodiment, the risk mitigation system 602 presents a computer-implemented apparatus for dynamically adapting the healthcare interventions in response to the continuously updated, multi-dimensional patient risk profile 144. Unlike traditional static systems that rely on predetermined treatment pathways, the computer-implemented risk mitigation system 602 is uniquely configured to synthesize, recalibrate, and personalize the treatment protocols in real-time based on fluctuations in a patient's real-time health risk status indicative through the risk profile 144 which is computer executable. The risk mitigation apparatus 602 may achieve this by integrating the risk profile acquisition interface 702 that may securely ingest the dynamic patient risk profile 144 from the risk assessment engine 100 shown in various figures, which may provide an aggregated clinical and social risk score. This real-time adaptation may facilitate managing high-risk patients with complex health profiles, where risk factors may fluctuate frequently due to ongoing changes in both clinical conditions and social determinants of health.

The risk mitigation system 602 may not only assess the risk factors but also adapt to changes in the patient's health status, generating the personalized healthcare interventions that may evolve alongside the patient risk. This risk mitigation apparatus 602 may provide an ability to autonomously recalibrate the personalized or patient-specific interventions based on real-time inputs, creating a self-learning healthcare management system that may adjust to patient needs without requiring constant human oversight. The system's framework is built around continuous feedback loops that may refine treatment actions based on patient outcomes, thereby improving precision and efficacy of subsequent interventions. The multi-dimensional patient risk profile 144 may facilitate in decision-making and may allow the risk mitigation system 602 to move healthcare beyond isolated data points and instead synthesize a holistic view of patient health. A seamless integration of the system 602 with external entities can allow multiple caregivers and health systems to participate in a coordinated, data-driven approach to patient care, fostering collaborative, accountable, and patient-centric healthcare based on continuously generated and monitored patient risk profile 144 through conducting risk intelligence.

The risk profile acquisition interface 702 may be configured to securely ingest and interpret the multi-dimensional patient risk profile 144. The multi-dimensional patient risk profile 144 or simply the risk profile 144 may include an aggregated clinical and social risk score to collectively indicate a dynamic patient's real-time health risk status. The risk profile acquisition interface 702 may serve as a primary conduit for receiving comprehensive risk data from a variety of healthcare data sources through the risk manager 120 or the risk assessment system 100, allowing for the integration of both clinical and non-clinical information into a unified risk assessment framework.

The risk profile acquisition interface 702 may operate under secure protocols to ensure that any sensitive patient data is protected throughout the acquisition process. The secure ingestion capability may be achieved through encryption and authentication mechanisms that may prevent unauthorized access and data tampering, thereby maintaining data integrity and confidentiality. The risk profile acquisition interface 702 may be further configured to interpret the ingested data by parsing, categorizing, and structuring various risk components according to predefined contextual rules.

In embodiments, the risk profile acquisition interface or simply interface 702 may be designed with Natural Language Processing (NLP) algorithms 802 that may extract valuable insights from unstructured clinical notes, such as physician observations, discharge summaries, and patient progress reports. The NLP algorithms 802 may analyze text data to identify relevant health conditions, symptoms, medication history, lifestyle factors, and other critical information that may not be captured in the structured data 106. The interface 702 may enable a comprehensive and accurate representation of the patient's multi-dimensional risk profile 144 by incorporating context-sensitive data from such as the clinical notes, ensuring that subtle yet significant insights are factored into risk assessments and mitigation.

The adaptive treatment protocol synthesizer 704 is a special purpose computer-implemented module in operative connection with the risk profile acquisition interface 702, wherein the adaptive treatment protocol synthesizer 704 may be configured to autonomously generate and recalibrate patient-specific treatment protocols in real-time. The adaptive treatment protocol synthesizer 704 may utilize data received from the multi-dimensional patient risk profile 144 through the risk profile acquisition interface 702 to continuously monitor and respond to variations within the patient's real-time health risk status. The adaptive treatment protocol synthesizer 704 may be specifically programmed to analyze changes in the multi-dimensional patient risk profile 144 and, in response to such variations, autonomously adjust and refine the one or more treatment protocols tailored to an individual patient.

The adaptive treatment protocol synthesizer 704 may operate by identifying the specific risk factors indicated through the multi-dimensional patient risk profile 144 and generating the targeted or personalized interventions aimed at mitigating these identified risk factors. The adaptive treatment protocol synthesizer 704 may leverage machine learning algorithms 804, a rule-based decision logic engine 806, and clinical guidelines and care pathways databases 808 to determine optimal treatment pathways, which may be dynamically recalibrated in accordance with the evolving patient data. Each patient-specific treatment protocol generated by the adaptive treatment protocol synthesizer 704 may be optimized to address a patient's unique health risks, thereby facilitating a personalized approach to risk mitigation. This configuration may allow the adaptive treatment protocol synthesizer 704 to maintain up-to-date treatment strategies that may be both responsive to real-time risk variations and effective in addressing specific health challenges presented by the individual patient's current risk profile 144.

The adaptive treatment protocol synthesizer 704 may include the rule-based decision logic engine 806, which may facilitate generating and refining the patient-specific treatment protocols. The rule-based decision logic engine 806 may communicate with the expert-defined clinical guidelines and established care pathways databases 808, which may provide a structured approach to managing various health conditions through computer implemented and computer executable processes and instruments. These guidelines and care pathways may be derived from reputable sources, such as clinical protocols, medical literature, and best practice guidelines and stored as computer executable data sources fetched through computer networked and implementable systems without human interventions.

As the multi-dimensional patient risk profile 144 is continuously updated, the rule-based decision logic engine or simply logic engine 804 used interchangeably without limitations may dynamically adjust the treatment protocols based on the most current data. For instance, if a patient's risk factors change due to fluctuating vital signs or social determinants of health, the logic engine 804 may update the treatment protocols to address the new risks. This dynamic adaptation may ensure that each patient's care is personalized, targeting their specific needs and optimizing the mitigation of identified risk factors and with automation utilizing computer-controlled systems and processes and without human intervention to avoid failure and risks occurring due to human errors and limitations.

In embodiments, the adaptive treatment protocol synthesizer 704 may include a patient-centric feedback mechanism 810 that may continuously incorporate self-reported health data and subjective experiences shared by the patient. This feedback, which can be input via patient portals or health apps and saved as executable files, may include information such as pain levels, side effects, and lifestyle behaviors. The adaptive treatment protocol synthesizer 704 may allow for personalized and patient-driven adjustments by integrating this self-reported data into the treatment protocols, thereby ensuring that the interventions align with the patient's ongoing experiences and preferences, thus enhancing the relevance and effectiveness of care.

The machine learning-driven next-best-action computational module 706 is a special purpose computer-implemented module in operative communication with the adaptive treatment protocol synthesizer 704. The next-best-action computational module 708 may be configured to analyze both the multi-dimensional patient risk profile 144, as acquired through the risk profile acquisition interface 702, and the one or more treatment protocols generated by the adaptive treatment protocol synthesizer 704. Through this operative connection, the next-best-action computational module 706 may utilize information provided by the adaptive treatment protocol synthesizer 704 to refine its analyses and determine subsequent patient-specific actions.

The machine learning-driven next-best-action computational module or next-best-action computational module or simply computational module 706 may employ machine learning algorithms 816 trained on historical patient data and outcomes 818 (including clinical guidelines, and prior treatment outcomes etc without limitations), enabling it to produce one or more hyper-personalized next-best actions 820 (or simply next best actions referred interchangeably) that may be specifically tailored to mitigate the identified risk factors within the patient's multi-dimensional risk profile 144. Each of these next-best actions 820 may be generated in real-time and dynamically adjusted to align with ongoing updates in the patient's risk profile 144, allowing the computational module 706 to respond to changes in patient status with precision. The next-best-actions computational module 706 may further incorporate predictive modeling techniques and predictive models 822 to anticipate future risk trajectories and adjust its output accordingly, ensuring that each generated next-best action is both proactive and responsive to the patient's current health risks.

The structural arrangement of the next-best-action computational module 706 in communication with the adaptive treatment protocol synthesizer 704 may allow for seamless integration and continuous recalibration of the patient-specific interventions. Upon receiving real-time updates from the adaptive treatment protocol synthesizer 704, the next-best-action computational module 706 may refine its next-best action recommendations to align with the latest treatment protocols 812 and variations in the multi-dimensional patient risk profile 144, ensuring that each recommended action is effectively targeted and continuously optimized for the patient's evolving risk factors and aimed to mitigate the patient risks thereby improving the patient risk profile 144.

The machine learning-driven next-best-action computational module 706 may leverage a suite of the predictive models 822 that may be trained on the extensive historical patient data 818, including clinical outcomes, treatment efficacy, and patient responses. Each predictive model of 818 may be designed to forecast a potential impact of the different interventions on a patient's health trajectory. The predictive models 822 may utilize a variety of data sources, such as clinical data, social determinants, and real-time health metrics, to generate a series of recommendations 824 that are machine readable and executable for the next best actions 820.

Each of the recommendations 824 may be weighted according to its predicted efficacy in reducing the patient's risk score. This may enable the risk mitigation system 602 to prioritize the interventions that are most likely to improve patient outcomes. For example, if the system 602 predicts that a specific treatment will have a higher impact on reducing the patient's risk score, that action may be recommended as the next-best action or actions 820 or part of the one or more next best actions 820. The learning algorithms 816 may continuously refine its predictions by incorporating feedback from past interventions, improving the accuracy and effectiveness of future recommendations.

The next-best-action computational module 706 may further incorporate a temporal decision-making model 826, which may allow it to account for both the current state of the patient's health and predicted future trajectories of risk. The temporal decision-making model 826 may consider how a patient's risk profile such as the risk profile 144 may evolve over time based on past health data, treatment responses, and external factors. The next-best-action computational module 706 may therefore generate the recommendations 824 by integrating temporal aspects, that are not only appropriate for the present moment but also proactive in addressing future risks.

For example, if the next-best-action computational module 706 predicts that a patient's condition may worsen in the next few days, the next-best action or actions 820 might involve an early intervention or preparation for future complications. This proactive decision-making may ensure that the treatment plans are optimized to prevent deterioration in the patient's health.

The next-best-action computational module 706 may continuously refine the recommendations 824 through feedback loops 828. Each time an intervention is executed, the next-best-action computational module 706 may monitor its impact on the patient's health and updates the learning algorithms 816 and the predictive models 822 based on the outcomes. This feedback from the feedback loops 828 may allow the next-best-action computational module 706 to adapt and improve the quality of its future recommendations, ensuring that treatment strategies are always tailored to the patient's current health status and the evolving risk profile 144.

The next-best-action computational module 706 may employ a causal inference algorithm 830 to identify underlying causal relationships between the various risk factors within the patient's multi-dimensional profile 144. Rather than merely addressing symptoms or correlating factors, the causal inference algorithm 830 may identify root causes that may contribute to specific risks, such as lifestyle choices or untreated conditions. This insight may enable the next-best-action computational module 706 to recommend the next-best actions 820 that not only mitigate visible symptoms but also address the underlying causes of the patient's risks, leading to more comprehensive and lasting risk reduction through collaborative care based on comprehensive and continuous risk intelligence.

The multi-entity collaboration engine 708 may be configured to facilitate secure, real-time bidirectional data exchange among two or more entities involved in the patient's care, thereby enabling a collaborative and synchronized approach to patient treatment. The multi-entity collaboration engine 708 may incorporate one or more tools 832 specifically designed to enable secure communication and data sharing across healthcare entities, such as hospitals, clinics, and individual healthcare providers, who may participate in managing and monitoring the patient's treatment holistically. The multi-entity collaboration engine 708 may utilize secure communication protocols or secure data exchange protocols 834, including data encryption, to ensure that all patient data, including the sensitive treatment protocols and the next-best actions, may be shared in a manner that preserves confidentiality and data integrity such as through blockchain. Through this secure data exchange, the multi-entity collaboration engine 708 may ensure that any updates to the one or more treatment protocols 812 generated by the adaptive treatment protocol synthesizer 704, as well as adjustments in the one or more next-best actions 820 produced by the machine learning-driven next-best-action computational module 706, are consistently visible and accessible to all authorized entities through appropriate access permissions.

Moreover, in some embodiments, the multi-entity collaboration engine 708 may be configured to present the risk score as a continuously visible metric, measurable on a continuous scale over time. This continuous display may allow each entity involved in the patient's care to observe changes in the patient's real-time risk profile 144, enhancing situational awareness and enabling timely, collaborative refinement of the treatment protocols 812 and the next-best actions 820 as the patient's condition evolves. The multi-entity collaboration engine 708 may enable a cohesive, data-driven approach to care by facilitating synchronization among all entities, ensuring that any intervention or adjustment to the patient's treatment is informed by the most recent risk score and collectively optimized based on real-time insights from all involved stakeholders and not in silos.

In an embodiment, the multi-entity collaboration engine 708 may incorporate the secure data exchange protocols based on blockchain technology, which may ensure that all data exchanges, including patient care actions, treatment updates, and risk profile modifications, are immutable, verifiable, and auditable. Blockchain may ensure that each action is recorded with a timestamp and that the data is cryptographically secured, preventing tampering or unauthorized changes.

The blockchain may allow the risk mitigation system 602 to create an auditable trail of all patient care activities, providing transparency and accountability in healthcare delivery. This is particularly important in healthcare settings where multiple providers may interact with a patient's care plan, ensuring that all actions are documented and verifiable.

The multi-entity collaboration engine 708 may be equipped with a real-time care coordination dashboard 836 that may visually represent the patient's multi-dimensional risk profile 144, treatment protocols 812, and progress in risk mitigation. The real-time care coordination dashboard 836 may present real-time data and actionable metrics, such as changes in risk scores, adjustments to the treatment protocols 812, and the outcomes of previous interventions.

The dashboard 836 may enable healthcare providers to monitor the patient's condition continuously and make timely adjustments to the care plan by providing real-time visual indicators. The dashboard 836 may also foster collaboration between multiple healthcare entities by offering a shared view of the patient's health status and treatment history.

In embodiments, the multi-entity collaboration engine 836 may include a real-time decision support system (not shown in figure) that may provide alerts and recommendations to healthcare providers based on an identified risk severity. When significant changes are detected in the patient's risk profile 144, such as a rise in vital signs that may indicate deterioration, the multi-entity collaboration engine 708 may generate real-time alerts. Each alert or recommendation may be contextually tailored, ensuring that the response is appropriate to the patient's current risk profile 144. For example, in cases of high-risk indicators, immediate intervention recommendations may be sent, while lower-risk changes might trigger recommendations for closer monitoring or non-urgent adjustments in care.

The risk mitigation system 602 may integrate several external tools such as the one or more tools 832 to improve the functionality and scope of patient risk management process. These tools 832 may include, but are not limited to, an Electronic Health Record (EHR) system 838, a Patient Relationships Management (PRM) system 840, a Patient Engagement System (PES) 842, and a Customer Relationships Management (CRM) system 844. The EHR system 838 may facilitate the secure and structured storage of a patient's medical history, laboratory results, diagnoses, and other clinical information. The EHR system 838 may contribute critical health data by interfacing with the multi-dimensional risk profile acquisition interface 702, which may be aggregated and continuously updated as part of the risk assessment process.

The PRM system 840 may support the monitoring of a patient's interactions and engagement with healthcare providers and associated computer systems, allowing for personalized communication and proactive care management. The PES system or simply PES 842 may integrate with the PRM system 840, enabling patients to participate in their care through self-reported data, such as lifestyle habits and satisfaction with treatment. The CRM system 844 may enable management of broader patient-provider relationships, tracking all communications, appointments, and follow-ups.

Each of these tools 832 may interface seamlessly with the risk mitigation system 602 or me be part of any of the components of the risk mitigation system 602 such as the risk profile acquisition interface 702 or the collaboration engine 708, in various embodiments without limitations, to provide a comprehensive, multidimensional view of the patient's health and engagement, which is essential for an effective generation of the personalized and adaptive treatment protocols 812. The multi-dimensional risk profile acquisition interface 702 may be designed to securely interface with the various healthcare data sources to collect and integrate clinical, social, and real-time data, which collectively form the comprehensive risk profile 144. These data sources have been discussed in conjunction with various figures.

In embodiments, the present invention provides the computer-implemented patient risk mitigation apparatus or system 602, structured to adaptively manage patient risks by dynamically calibrating the healthcare interventions in response to continuously evolving the multi-dimensional patient risk profiles 144. The computer-implemented patient risk mitigation apparatus 602 may be implemented through a series of interrelated components, each performing specialized functions for real-time risk assessment, adaptive treatment generation, and collaborative care management, designed to improve healthcare precision and responsiveness. The following describes sequential operational steps, without limitations, which may be performed in same or different sequence.

The process begins by initializing the risk assessment system 100, configured to generate an initial scoring of the patient-specific risks. This scoring process may involve secure acquisition of clinical and social data inputs from external data sources, including but not limited to the electronic health records (EHR) 108, the social determinants of health (SDoH) 128 related databases, and the real-time patient monitoring devices 112. The risk assessment system 100 may aggregate these diverse data inputs into a comprehensive initial risk score, which reflects the patient's overall risk level upon activation of the risk assessment system 100. This risk score may provide a baseline against which future changes may be detected and evaluated, establishing a dynamic starting point for ongoing risk assessment.

Following the initial risk scoring, the risk assessment system 100 may set baseline values and thresholds specific to each patient's risk profile 144. The baseline values may be determined based on the data acquired from such as without limitations historical patient records, clinical guidelines, and individualized health parameters, enabling a tailored risk model for each patient. These baselines may represent acceptable ranges for each critical parameter and may allow for detection of deviations indicative of increased health risks. Thresholds for alert generation may be predefined but can be adjusted based on patient response over time, supporting a patient-specific approach to risk tolerance.

Upon establishing the baseline values, the risk assessment system 100 may actively monitor for the deviations in the patient health status by continuously comparing incoming real-time data with the established thresholds. This deviation detection mechanism may operate autonomously, scanning for any shifts in the risk parameters or risk scores that may exceed tolerance levels defined during baseline configuration. When such deviations are detected, the risk assessment system 100 may automatically generate an alert, which may be transmitted to authorized healthcare providers and caregivers involved in the patient's treatment or to their associated computer systems entirely through machine executable infrastructures. This alert may indicate a potential escalation in risk, facilitating timely medical intervention and adjustment to the treatment protocols 812.

In response to the detected deviations, the risk assessment system 100 may initiate a dynamic weighting adjustment protocol. This may enable real-time reallocation of weighting across various the risk factors or risk parameters or risk scores within the patient's profile 144, assigning higher weighting to critical parameters based on their current risk level. For instance, if a cardiovascular parameter (e.g., blood pressure or heart rate) exhibits a pattern that diverges significantly from baseline, the system 100 may dynamically increase its weighting, thereby prioritizing its influence on the risk profile 144 and subsequent treatment decisions. The dynamic weighting adjustment may allow the system 100 to adaptively prioritize the risk factors according to the patient's present health status, improving precision of risk assessment and intervention planning.

Following the dynamic adjustment of risk weightings, the risk assessment system 100 may recalibrate the patient's risk profile 144 to incorporate real-time changes in the health data. This recalibration process may involve recalculating the aggregated risk score based on updated weighting factors, ensuring that the patient's current risk profile 114 accurately reflects their health status. The recalibrated profile 144 may serve as the basis for the generation of the adaptive treatment protocols 812, enabling the system 100 to provide timely, individualized responses to each patient's evolving health needs. This recalibrated risk profile 144 may be continuously updated, allowing the system 100 to maintain an accurate reflection of the patient's risk levels over time.

Based on the recalibrated risk profile 144, the risk mitigation system 602 may generate the patient-specific treatment protocols or simply treatment protocols 812. The treatment protocols 812 may be formulated by integrating data from clinical guidelines, patient-specific health records, social determinants of health, and predictive modeling algorithms. In conjunction with this, the risk mitigation system 602 may evaluate the patient's risk profile 144 and the associated treatment protocols 812, generating the recommended next best actions 820 tailored to address the risk factors. Each recommended action or actions 820 may be weighted according to predicted efficacy, ensuring a prioritized approach to risk mitigation. These next best actions 820 may be dynamically adjusted to reflect updates in the patient's risk profile 144, maintaining alignment with the real-time patient health status.

The risk mitigation system 602 may be further equipped with the multi-entity collaboration engine 708, which may facilitate real-time data exchange and collaborative decision-making among multiple healthcare providers involved in the patient's care. This may allow using the secure data exchange protocols 834, including but not limited to blockchain technology, ensuring that all patient care actions are logged in an immutable and verifiable format. Through a shared care coordination dashboard such as the dashboard 836, authorized entities can access real-time updates to the patient's risk profile 144, monitor treatment progress, and collaboratively refine care strategies as needed. This collaborative environment may support the continuous progression of risk mitigation through coordinated and precise care interventions, enhancing patient outcomes by adapting interventions based on both individual provider input and system-driven risk assessments using special purpose machine-based systems that facilitate automation including such as hardware components and devices etc.

The present invention may further provide a method for intervention tracking and follow-up, that may be designed to improve the ability of the risk mitigation system 602 to mitigate patient risks by incorporating the targeted interventions and analyzing their outcomes over time. Upon identifying specific risks, the system 602 may generate one or more intervention flags at predefined intervals. These flags may represent a technical strategy within the broader framework of feedback loops and may be activated through two primary mechanisms: human intervention and automated business logic. Human-triggered flags may arise from healthcare providers detecting significant changes in a patient's condition, while automated flags may rely on algorithms embedded in software or hardware to identify real-time deviations in patient data from baseline values.

The interventions facilitated by the system 602 may vary in complexity. Complex interventions, such as surgical procedures or prescribed medications, may address heightened risks through clinical decision-making, often integrating with the next-best-action recommendations or next best actions 820. Simpler interventions, such as educational or training sessions delivered by non-clinicians, may be provided through actionable recommendations tailored to the dynamic risk profiles 144. Th interventions may aim to address risks efficiently and may form an integral component of adaptive design of the system 602.

A key aspect of this invention is its ability to track the interventions and analyze their effects on the continuous risk profile 144 over defined timeframes. The system 602 may evaluate whether an intervention results in an increase, decrease, or no discernible change in the patient's risk profile 144. This analytical process forms a feedback loop that may allow the system 602 to refine its recommendations, ensuring more precise and effective guidance for future interventions. The system may dynamically adjust to improve risk mitigation and automated patient care by continuously learning from outcomes of the intervention.

The system may in some embodiments include an intervention tracking module 710 (shown in FIG. 7), which may log the interventions, categorize them (e.g., medical procedures, medication prescriptions, or non-clinical educational sessions), and assess their impact on the risk profile 144. Findings from the intervention tracking module 710 may be fed into the next-best-action computational module 706, enabling adjustments based on observed intervention outcomes. This interplay may allow responsiveness in the system 602 that may integrate prior learning into future recommendations.

The system 602 may further introduce a mechanism to trigger intervention flags at specific intervals. These triggers may stem from human observations, such as a clinician noting critical changes, or automated detection of significant deviations in patient metrics by embedded business logic. This dual approach may ensure comprehensive monitoring and timely responses to evolving patient conditions, thereby improving the reliability and adaptability of the system 602. The system 602 may optimize intervention strategies through its continuous feedback loop and robust analytical capabilities, addressing the risks effectively and promoting improved patient outcomes.

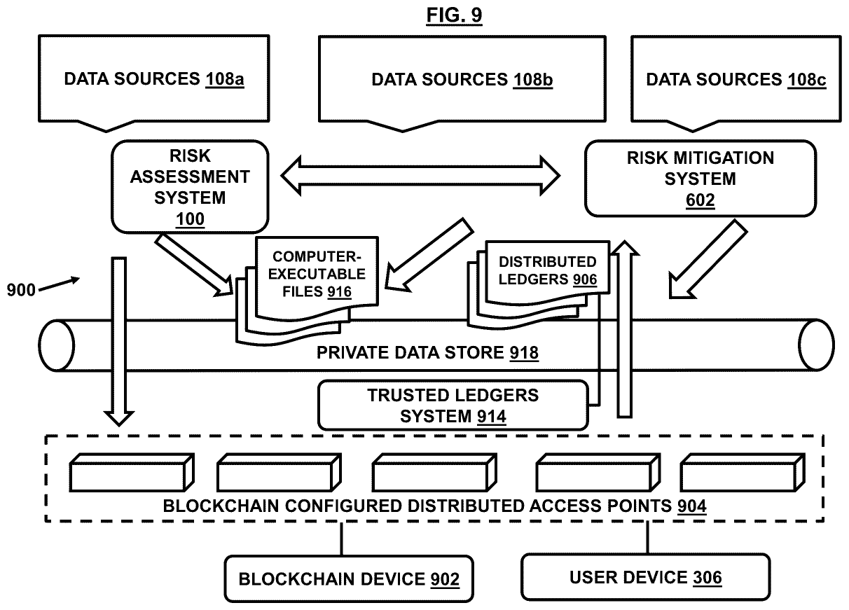

FIG. 9, with reference to FIGS. 1 through 8, illustrates an exemplary blockchain-configured ecosystem architecture 900 containing one or more components of the patient risk mitigation system 602 and the risk assessment system 100, and additional components to ensure the integrity of transactions and digital data (including information blocks and precision blocks) shared/processed during data transfer or storage, as previously discussed in this document. The blockchain-configured ecosystem architecture 900 provides a crowdsourced integrity network for storing data accessed, extracted, or transformed, for sharing or storing across a computer network. This approach contrasts with information stored locally by various participants or databases, which may be vulnerable to tampering.

The ecosystem architecture 900 may be blockchain-configured to incorporate various blockchain devices. For instance, the risk assessment system 100 and the risk mitigation system 602 may interact with a blockchain device 902 through a plurality of blockchain-configured distributed access points 904. A network facilitating interaction among all components may be a blockchain integrity network, fostering trust among various participants, entities, systems, and components thereof, and their associated computing terminals or devices, even if the devices/terminals or machines do not have direct knowledge of one another. The blockchain network enables secure connections, transactions, recording, and sharing of data, information blocks, precision blocks, and tokens, including service and authorization tokens, through a trusted system. The blockchain maintains a record of transactions, data exchanges, and updates from various terminals/devices on distributed ledgers 906, which provides a tamper-resistant history to build trust among terminals/devices (such as those associated with various participants or nodes) enabling peer-to-peer or peer-to-client interactions through a distributed digital ledger technology system. The ecosystem architecture 900 includes a distributed trusted ledger system 914 containing the distributed blockchain ledgers 906, which are associated with a plurality of computing terminals and devices. Each ledger may store a copy of computer-executable files 916, which contain data inputs related to the multi-dimensional patient risk profile 144, the adaptive treatment protocols 812, and the next-best actions 820, and other information relevant to patient risk mitigation. This distributed ledger system 914 allows for rule-based contracts that execute when specified conditions are met, enabling automated adjustments to the treatment protocols 812 based on real-time data changes.

The various computing terminals or devices in the network serve as distributed peer-to-peer nodes and connections. The risk assessment system 100 and the risk mitigation system 602 and components thereof may be configured to process context inputs and information blocks through the blockchain network based on defined rules. Each terminal/device/node in the ecosystem architecture 900 may receive a copy of the blockchain, which may be automatically downloaded upon joining the blockchain integrity network, thereby decentralizing the network. Every permissioned node or device in the network may act as an administrator of the blockchain, participating voluntarily in the decentralized network.

The blockchain mitigates risks associated with centralized data storage by distributing data across the network, which may include the computer-executable files 916 containing information blocks pertaining to patients etc, context inputs, context patterns, and various tokens/codes, including transaction codes. Security on the blockchain is achieved using encryption technology and validation mechanisms, which ensure data integrity. Public and private keys facilitate secure access, where a public key defines a user's address on the blockchain, and a private key enables access to digital assets within the network.

In an embodiment, the distributed ledgers 906 enable the coding of smart contracts (using smart contract systems) that execute upon the satisfaction of predefined conditions. These smart contracts may protect various data pieces associated with patient care and mitigate risks of unauthorized file copying and redistribution, ensuring patient privacy rights are maintained.

The blockchain-configured ecosystem architecture 900 may provide a private view for various devices and entities operating in the network through the private data store 918. This allows each permissioned device, such as the risk assessment system 100 or the risk mitigation system 602, to privately access the computer-executable files 916 associated with specific tasks, user inputs, and care directives based on permissions granted according to each entity's identity. The risk assessment system 100 or the risk mitigation system 602 may access these files through the private data store 918 via the distributed blockchain-configured access points 904, where each access point supports multi-party access based on defined rights.

The private data store 918 offers virtual storage to facilitate secure interaction, information exchange, review, and presentation of the computer-executable files 916. For example, the private data store 918 may allow the virtual presentation of only limited portions of executable files to specific entities, respecting permissions for access and review. The private data store 918 may be configured to auto-hash review interactions at defined intervals, ensuring that the computer-executable files 916 remain secure and private per access rights authorized to each node, while maintaining synchronization with permissioned access.

In an embodiment, the blockchain-configured digital ecosystem architecture 900 may incorporate a federated blockchain consisting of several entities/participants (including healthcare providers, administrators, and patients) and their associated devices. These components jointly interact to process data transfers through a trusted, secure, and distributed network of blockchain-configured access points 904.

In an embodiment, the blockchain device 902 may include the distributed trusted ledger system 914 containing the distributed blockchain ledgers 906 associated with the computing terminals. Each ledger stores a copy of computer-executable files that include data about the patient risk profile 144, the treatment protocols 812 and the next best actions 820, next-best action evidence, and data consumption information based on respective genuineness scores. When stored alongside these scores, this information provides a verifiable record of interactions within the patient risk mitigation process, supporting the system's objectives by ensuring data integrity and transparency through the blockchain.

Several numbered examples are listed below in accordance with various embodiment.

Example 1: A system for monitoring glucose concentration and generating a continuous and adaptive patient risk profile, the system comprising: a continuous glucose sensor configured to continuously measure the glucose concentration and output a data stream associated with the glucose concentration; a device operatively connected to the continuous glucose sensor, the device comprising at least one processor and memory, the memory storing instructions that, when executed by the processor, cause the system to retrieve and integrate the data stream from the continuous glucose sensor with other patient data, including data from one or more external sources; process the patient data using normalization and validation techniques; and generate a continuous patient risk profile based on a real-time risk scoring algorithm that integrates glucose concentration data and patient data from various sources.

Example 2: The of example 1, wherein the at least one processor is further configured to apply a dynamic weighting system to the patient data, adjusting the contribution of each data source based on factors including one or more of data reliability and relevance.

Example 3: The system of example 1 wherein the at least one processor is further configured to continuously monitor and recalibrate the patient risk profile based on deviations in the patient data, including the glucose concentration data, adjusting the risk profile based on a magnitude and persistence of the deviations.

Example 4: The system of example 1, further comprising a user interface configured to display the glucose concentration data and the continuously updated patient risk profile.

Example 5: The system of example 1, wherein the device is configured to toggle between a first mode for displaying real-time glucose concentration values associated with the continuous glucose sensor and a second mode for displaying the patient's risk profile, wherein the toggling is controlled automatically based on predefined conditions such as significant changes in glucose concentration beyond a threshold or patient risk profile deviations, or manually through a user input.

Example 6: The system of example 5, wherein the at least one processor is configured to trigger an alert based on the changes in the glucose concentration or the patient risk profile deviations, wherein the alert prompts the device to automatically switch to the mode displaying the most critical information.

Example 7: A system for monitoring glucose concentration and generating a continuous and adaptive patient risk profile based on the glucose concentration, the system comprising: a continuous glucose sensor configured to continuously measure glucose concentration and output a data stream associated with glucose concentration; a device configured to operatively connect with the continuous glucose sensor, the device comprising a single-point glucose monitor configured to provide discrete glucose concentration measurements, at least one processor and a memory operatively connected to the processor, the memory storing instructions that, when executed by the processor, cause the system to: retrieve and integrate the data stream from the continuous glucose sensor with patient data from structured data from Electronic Health Records (EHRs), semi-structured data from monitoring devices, and unstructured data from external sources; subject each category of patient data to a source-specific normalization and validation process prior to integration;

generate a continuous and adaptive patient risk profile by executing a real-time risk scoring algorithm, wherein the patient risk profile is derived from the aggregated and pre-processed patient data, including glucose concentration data from the continuous glucose sensor; adjust the applied weighting in real time based on detected deviations from baseline values within the glucose concentration data stream and other patient data; a user interface configured to display measured glucose concentration values associated with both the single-point glucose monitor and the continuous glucose sensor, along with the patient risk profile generated by the system.

Example 8: A computer-implemented risk mitigation system configured to continuously assess and adaptively mitigate risks within a digital environment, computer-implemented risk mitigation system comprising: a dynamic risk intelligence engine that continuously collects and integrates diverse streams of patient data from multiple sources, including real-time monitoring devices, electronic health records (EHR), socio-environmental data, and patient-reported information, wherein the risk intelligence engine: employs machine learning algorithms to identify, in real-time, patient-specific risk indicators across medical, social, and environmental dimensions, and generates a continuously adaptive risk profile that reflects both immediate and forecasted health risks, updated iteratively based on incoming data; executes dynamic, personalized risk mitigation protocols based on the adaptive risk profile, automatically selecting and initiating intervention actions that are contextually suited to the identified risk indicators, and modifies the intervention actions in real-time according to detected changes in the patient's risk profile, prioritizing responses based on urgency and anticipated outcomes to coordinate patient-centered risk reduction efforts.

The various components described herein and/or illustrated in the figures may be embodied as hardware-enabled modules and may be a plurality of overlapping or independent electronic circuits, devices, and discrete elements packaged onto a circuit board to provide data and signal processing functionality within a computer. An example might be a comparator, inverter, or flip-flop, which could include a plurality of transistors and other supporting devices and circuit elements. The modules that include electronic circuits process computer logic instructions capable of providing digital and/or analog signals for performing various functions as described herein. The various functions can further be embodied and physically saved as any of data structures, data paths, data objects, data object models, object files, database components. For example, the data objects could include a digital packet of structured data. Example data structures may include any of an array, tuple, map, union, variant, set, graph, tree, node, and an object, which may be stored and retrieved by computer memory and may be managed by processors, compilers, and other computer hardware components. The data paths can be part of a computer CPU that performs operations and calculations as instructed by the computer logic instructions. The data paths could include digital electronic circuits, multipliers, registers, and buses capable of performing data processing operations and arithmetic operations (e.g., Add, Subtract, etc.), bitwise logical operations (AND, OR, XOR, etc.), bit shift operations (e.g., arithmetic, logical, rotate, etc.), complex operations (e.g., using single clock calculations, sequential calculations, iterative calculations, etc.). The data objects may be physical locations in computer memory and can be a variable, a data structure, or a function. Some examples of the modules include relational databases (e.g., such as Oracle® relational databases), and the data objects can be a table or column, for example. Other examples include specialized objects, distributed objects, object-oriented programming objects, and semantic web objects. The data object models can be an application programming interface for creating HyperText Markup Language (HTML) and Extensible Markup Language (XML) electronic documents. The models can be any of a tree, graph, container, list, map, queue, set, stack, and variations thereof, according to some examples. The data object files can be created by compilers and assemblers and contain generated binary code and data for a source file. The database components can include any of tables, indexes, views, stored procedures, and triggers.

In an example, the embodiments herein can provide a computer program product configured to include a pre-configured set of instructions, which when performed, can result in actions as stated in conjunction with various figures herein. In an example, the pre-configured set of instructions can be stored on a tangible non-transitory computer readable medium. In an example, the tangible non-transitory computer readable medium can be configured to include the set of instructions, which when performed by a device, can cause the device to perform acts similar to the ones described here.

The embodiments herein may also include tangible and/or non-transitory computer-readable storage media for carrying or having computer-executable instructions or data structures stored thereon. Such non-transitory computer readable storage media can be any available media that can be accessed by a general purpose or special purpose computer, including the functional design of any special purpose processor as discussed above.

By way of example, and not limitation, such non-transitory computer-readable media can include RAM, ROM, EEPROM, CD-ROM or other optical disk storage, magnetic disk storage or other magnetic storage devices, or any other medium which can be used to carry or store desired program code means in the form of computer-executable instructions, data structures, or processor chip design. When information is transferred or provided over a network or another communications connection (either hardwired, wireless, or combination thereof) to a computer, the computer properly views the connection as a computer-readable medium. Thus, any such connection is properly termed a computer-readable medium. Combinations of the above should also be included within the scope of the computer-readable media.

Computer-executable instructions include, for example, instructions and data which cause a special purpose computer or special purpose processing device to perform a certain function or group of functions. Computer-executable instructions also include program modules that are executed by computers in stand-alone or network environments. Generally, program modules include routines, programs, components, data structures, objects, and the functions inherent in the design of special-purpose processors, etc. that perform particular tasks or implement particular abstract data types. Computer-executable instructions, associated data structures, and program modules represent examples of the program code means for executing steps of the methods disclosed herein. The particular sequence of such executable instructions or associated data structures represents examples of corresponding acts for implementing the functions described in such steps.

The techniques provided by the embodiments herein may be implemented on an integrated circuit chip (not shown). The chip design is created in a graphical computer programming language, and stored in a computer storage medium (such as a disk, tape, physical hard drive, or virtual hard drive such as in a storage access network. If the designer does not fabricate chips or the photolithographic masks used to fabricate chips, the designer transmits the resulting design by physical means (e.g., by providing a copy of the storage medium storing the design) or electronically (e.g., through the Internet) to such entities, directly or indirectly. The stored design is then converted into the appropriate format (e.g., GDSII) for the fabrication of photolithographic masks, which typically include multiple copies of the chip design in question that are to be formed on a wafer. The photolithographic masks are utilized to define areas of the wafer (and/or the layers thereon) to be etched or otherwise processed.

The resulting integrated circuit chips can be distributed by the fabricator in raw wafer form (that is, as a single wafer that has multiple unpackaged chips), as a bare die, or in a packaged form. In the latter case the chip is mounted in a single chip package (such as a plastic carrier, with leads that are affixed to a motherboard or other higher level carrier) or in a multichip package (such as a ceramic carrier that has either or both surface interconnections or buried interconnections). In any case the chip is then integrated with other chips, discrete circuit elements, and/or other signal processing devices as part of either (a) an intermediate product, such as a motherboard, or (b) an end product. The end product can be any product that includes integrated circuit chips, ranging from toys and other low-end applications to advanced computer products having a display, a keyboard or other input device, and a central processor.

Furthermore, the embodiments herein can take the form of a computer program product accessible from a computer-usable or computer-readable medium providing program code for use by or in connection with a computer or any instruction execution system. For the purposes of this description, a computer-usable or computer readable medium can be any apparatus that can comprise, store, communicate, propagate, or transport the program for use by or in connection with the instruction execution system, apparatus, or device.

The medium can be an electronic, magnetic, optical, electromagnetic, infrared, or semiconductor system (or apparatus or device) or a propagation medium. Examples of a computer-readable medium include a semiconductor or solid-state memory, magnetic tape, a removable computer diskette, a random access memory (RAM), a read-only memory (ROM), a rigid magnetic disk and an optical disk. Current examples of optical disks include compact disk-read only memory (CD-ROM), compact disk-read/write (CD-R/W) and DVD.

A data processing system suitable for storing and/or executing program code will include at least one processor coupled directly or indirectly to memory elements through a system bus. The memory elements can include local memory employed during actual execution of the program code, bulk storage, and cache memories which provide temporary storage of at least some program code in order to reduce the number of times code must be retrieved from bulk storage during execution.

Input/output (I/O) devices (including but not limited to keyboards, displays, pointing devices, etc.) can be coupled to the system either directly or through intervening I/O controllers. Network adapters may also be coupled to the system to enable the data processing system to become coupled to other data processing systems or remote printers or storage devices through intervening private or public networks. Modems, cable modem and Ethernet cards are just a few of the currently available types of network adapters.

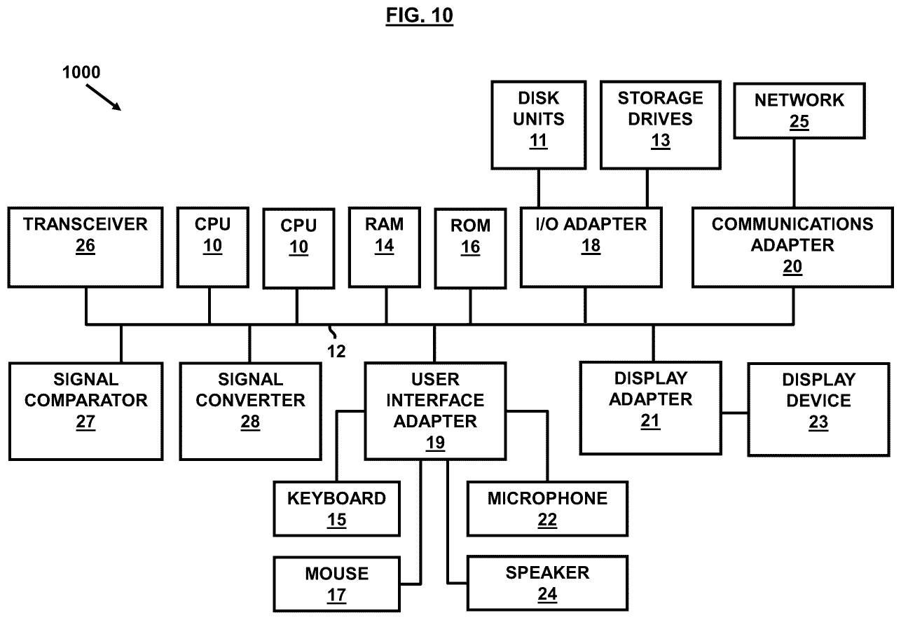

A representative hardware environment for practicing the embodiments herein is depicted in FIG. 10, with reference to FIGS. 1 through 9. This schematic drawing illustrates a hardware configuration of an information handling/computer system 1000 in accordance with the embodiments herein.

The system 1000 comprises at least one processor or central processing unit (CPU) 10. The CPUs 10 are interconnected via system bus 12 to various devices such as a random access memory (RAM) 14, read-only memory (ROM) 16, and an input/output (I/O) adapter 18. The I/O adapter 18 can connect to peripheral devices, such as disk units 11 and tape drives 13, or other program storage devices that are readable by the system. The system 1000 can read the inventive instructions on the program storage devices and follow these instructions to execute the methodology of the embodiments herein. The system 1000 further includes a user interface adapter 19 that connects a keyboard 15, mouse 17, speaker 24, microphone 22, and/or other user interface devices such as a touch screen device (not shown) to the bus 12 to gather user input. Additionally, a communication adapter 20 connects the bus 12 to a data processing network, and a display adapter 21 connects the bus 12 to a display device 23 which may be embodied as an output device such as a monitor, printer, or transmitter, for example. Further, a transceiver 26, a signal comparator 27, and a signal converter 28 may be connected with the bus 12 for processing, transmission, receipt, comparison, and conversion of electric or electronic signals.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the present invention.

What is claimed is:

1. A computer-implemented system for generating a continuous and adaptive patient risk profile through multi-source data integration, the system comprising:
a processor; and
a memory operatively connected to the processor, the memory storing instructions that, when executed by the processor, cause the system to:
retrieve and integrate patient data from structured data from Electronic Health Records (EHRs), semi-structured data from monitoring devices, and unstructured data from external sources, wherein each category of patient data is subjected to a source-specific normalization and validation process prior to integration;
generate a patient risk profile by executing a real-time risk scoring algorithm, wherein the patient risk profile is derived from the aggregated and pre-processed patient data;
apply a dynamic weighting to each type of patient data, said weighting being determined by predefined contextual rules, wherein said contextual rules incorporate reliability factor of the data source and relevance of the data to the patient's current health context in determining the weighting;
adjust the applied weighting in real-time based on detected deviations from baseline values within the patient data; and
monitor and recalibrate the patient risk profile continuously at predefined time intervals, wherein the frequency and timing of recalibration are dynamically adjusted based on a duration and magnitude of deviations in the patient data from baseline values.

2. The system of claim 1, wherein the source-specific normalization and validation process for unstructured and semi-structured data incorporates a machine learning model trained specifically on diverse data patterns, including environmental, social, and behavioral data, to perform adaptive validation of data based on contextual relevance to the patient's current health status, the model executed by a hardware-based AI processing unit.

3. The system of claim 2, wherein the source-specific normalization and validation process for integrating the patient data from the unstructured sources further comprises: a source-specific pre-processing module configured to retrieve and validate Social Determinants of Health (SDoH) data from the external databases and social media platforms using a machine-learning-based natural language processing (NLP) engine, wherein the machine-learning-based NLP engine normalizes the unstructured data comprising the SDoH by converting the unstructured data into structured formats compatible with the real-time risk scoring algorithm.

4. The system of claim 3, wherein the machine-learning-based NLP engine assigns a reliability factor to the unstructured data based on an accuracy and timeliness of the retrieved SDoH data, the reliability factor being dynamically adjusted based on real-time evaluations of data integrity and relevance to the patient's current health context.

5. The system of claim 4, wherein the machine-learning-based NLP engine is further configured to assign the reliability factor to the unstructured data by:
parsing and extracting key entities from the unstructured data using named entity recognition (NER) and other NLP techniques;
applying a machine learning model trained on labeled datasets to evaluate the accuracy of the extracted data by comparing it against known, validated sources;
determining the timeliness of the unstructured data by analyzing timestamps and comparing it to current patient health events; and
calculating the reliability factor based on a weighted combination of accuracy and timeliness, the reliability factor being used to adjust the weighting of the unstructured data when calculating the patient's risk profile.

6. The system of claim 1, comprising a real-time data monitoring module configured to continuously track and collect the patient data from one or more of the monitoring devices, the external sources, and the EHRs, the real-time data monitoring module being integrated with the processor and using a hardware-based processing unit to dynamically adjust the patient's risk profile in response to the detected deviations in the patient data from the baseline values, wherein the recalibration occurs without human intervention for generating a continuous comprehensive risk profile.

7. The system of claim 1, comprising a predictive health event detection engine implemented on the processor, wherein the predictive health event detection engine is configured to continuously analyze patient data trends and generate an alert when the risk profile predicts an upcoming adverse health event, the predictive health event detection engine using one or more machine learning model trained on historical patient data to detect early warning signs of critical health deviations.

8. The system of claim 7, comprising a generative AI engine operatively connected to the processor, wherein the generative AI engine is configured to generate one or more hypothetical patient risk profiles by simulating future health scenarios through predictive modeling, the predictive modeling based on the patient data trends, historical deviations, and stochastic analysis of evolving health patterns across the structured data, the semi-structured data, and the unstructured data.

9. The system of claim 1, wherein the patient data retrieved by the system comprises one or more computer-executable files containing the structured data, semi-structured data, and the unstructured data of various types, the computer-executable files including one or more of:
patient health data from the Electronic Health Records (EHRs);
Social Determinants of Health (SDoH) data from the external databases;
wearable devices data monitoring patient health metrics;
patient-reported information entered through a user interface;
social media data analyzed for behavioral and lifestyle insights; and
hereditary health data retrieved from family health records,
wherein each computer-executable file is retrieved by a secure data retrieval device and processed by the processor to normalize, validate, and integrate the patient data for real-time risk profile generation.

10. The system of claim 1, wherein the local assistant hub comprises a modular hardware architecture that includes one or more interchangeable communication modules for supporting multiple wireless protocols, including one or more of Wi-Fi, Bluetooth, ZigBee, and Thread, configured to enable integration of the Electronic Health Records (EHRs), the monitoring devices, and the external sources using one or more communication standards without requiring a hardware upgrade.

11. The system of claim 10, wherein the local assistant hub includes a data encryption module for ensuring secure local storage and transmission of the patient data, wherein the encryption module is configured to operate without requiring continuous connection to an external server, ensuring patient data privacy.

12. The system of claim 11, wherein the local assistant hub further comprises a local processing unit for performing on-device data analytics and real-time risk calculations, thereby reducing latency and minimizing reliance on cloud-based processing for continuous monitoring of the risk.

13. The system of claim 12, wherein the local assistant hub is configured to support data integration from the monitoring devices, wherein the monitoring devices comprises one or more of a heart rate monitor, a continuous glucose monitor, a sleep tracking device, wherein each of the monitoring devices is configured to communicate with the local assistant hub using a device-specific protocol and transmit the patient data in real-time.

14. The system of claim 13, wherein the semi structured data comprises a patient-generated information, further wherein the local assistant hub includes an adaptive interface for receiving the patient-generated information via one or more through voice, touch, or text input, allowing a user to manually input computer executable details into the system for integration into the patient risk profile.

15. The system of claim 14, wherein the local assistant hub is equipped with natural language processing (NLP) capabilities to interpret the patient-generated information, including voice commands, and translate these into actionable data points that are integrated into the patient risk profile.

16. The system of claim 10, wherein the local assistant hub includes a housing containing a display interface for providing real-time feedback on the patient risk profile, wherein the interface displays aggregated patient data including the structured data from the Electronic Health Records (EHRs), the semi-structured data from the monitoring devices, and the unstructured data from the external sources in a consolidated view for interpretation by a user.

17. A computer-implemented method for generating a continuous and adaptive patient risk profile through multi-source data integration, the method comprising:
retrieving and integrating patient data from structured data from Electronic Health Records (EHRs), semi-structured data from monitoring devices, and unstructured data from external sources, wherein each category of patient data is subjected to a source-specific normalization and validation process prior to integration;
generating a patient risk profile by executing a real-time risk scoring algorithm, wherein the patient risk profile is derived from the aggregated and pre-processed patient data;
applying a dynamic weighting to each type of patient data, wherein said weighting is determined by predefined contextual rules, said rules incorporating a reliability factor of the data source and relevance of the data to the patient's current health context;
adjusting the applied weighting in real-time based on detected deviations from baseline values within the patient data; and
monitoring and recalibrating the patient risk profile continuously at predefined time intervals, wherein the frequency and timing of recalibration are dynamically adjusted based on the duration and magnitude of deviations in the patient data from baseline values.

18. The method of claim 17, comprising:
analyzing patient data trends continuously using a predictive health event detection engine, wherein the engine uses at least one machine learning model trained on historical patient data to detect early warning signs of critical health deviations;
generating an alert when the predictive health event detection engine determines, based on the patient risk profile, that an adverse health event is likely to occur; and
adjusting the patient risk profile in real time in response to the alert, said adjustment being performed by recalculating the patient's risk profile based on the detected trends and predicted health event.

19. The method of claim 17, comprising:
assigning a reliability factor to the unstructured data retrieved from Social Determinants of Health (SDoH) data sources using a machine-learning-based Natural Language Processing (NLP) engine, wherein the reliability factor is determined based on the accuracy and timeliness of the retrieved data;
dynamically adjusting the reliability factor in real time based on one or more predefined parameters; and
using the dynamically adjusted reliability factor to modify the weighting applied to the unstructured data when calculating the patient risk profile.

20. A computer-implemented patient risk mitigation apparatus for continuously adapting healthcare interventions in response to a dynamic multi-dimensional patient risk profile, the computer-implemented patient risk mitigation apparatus comprising:
a risk profile acquisition interface configured to securely ingest and interpret the multi-dimensional patient risk profile from an external risk assessment engine, wherein the multi-dimensional patient risk profile includes an aggregated clinical and social risk score to collectively indicate a dynamic patient's real-time health risk status;
an adaptive treatment protocol synthesizer in operative connection with the risk profile acquisition interface, configured to autonomously generate and recalibrate patient-specific one or more treatment protocols in real-time in response to variations in the received multi-dimensional patient risk profile, the one or more patient treatment protocols being specifically targeted at mitigating identified one or more risk factors indicative through the multi-dimensional patient risk profile; and
a machine learning-driven next-best-action computational module, in communication with the adaptive treatment protocol synthesizer, configured to analyze the multi-dimensional patient risk profile and the one or more treatment protocols, and produce one or more hyper-personalized next best actions tailored to address the identified one or more risk factors, with each of the one or more next best actions dynamically adjusted to align with updates in the multi-dimensional patient risk profile.
